(19) United States
(12) Patent Application Publication
(10) Pub. No.: US 2010/0219085 A1
(43) Pub. Date: Sep. 2, 2010

(54) Analyte Sensor Offset Normalization

(75) Inventor: Henry W. Oviatt, JR., Temecula, CA (US)

Correspondence Address: EDWARDS LIFESCIENCES CORPORATION, LEGAL DEPARTMENT, ONE EDWARDS WAY, IRVINE, CA, 92614, US

(73) Assignee: Edwards Lifesciences Corporation, Irvine, CA (US)

(21) Appl. No.: 12/701,829

(22) Filed: Feb. 8, 2010

Related U.S. Application Data
(60) Provisional application No. 61/156,170, filed on Feb. 27, 2009.

(51) Int. Cl. G01N 27/26; G01N 33/50

(52) U.S. Cl. 205/782; 204/406; 205/792

(57) ABSTRACT

Measurements of current from a working electrode and current from a blank electrode are received and a ratio corresponding to the ratio of surface areas of the working electrode and the blank electrode is determined. This ratio is used to correct any differential current offset between an analyte (working) electrode and a control (blank) electrode to yield a more accurate net current output. Systems, methods and computer program products are further described for measuring an analyte concentration disclosed are for calculating the amount of an analyte in a fluid using a biosensor.

### CLAIM OF PRIORITY UNDER 35 U.S.C. §119

The application claims the benefit of U.S. Provisional Application No. 61/156,170 filed Feb. 27, 2009, entitled “Analyte Sensor Offset Normalization” and assigned to the assignee hereof and hereby incorporated by reference in its entirety.

### BACKGROUND

1. Field of the Invention

The invention relates generally to analyte measuring systems, methods and computer program products.

2. Description of Related Art

Controlling blood glucose levels for diabetics and other patients can be a vital component in critical care, particularly in an intensive care unit (ICU), operating room (OR), or emergency room (ER) setting where time and accuracy are essential. Presently, one of the most reliable way to obtain a highly accurate blood glucose measurement from a patient is by a direct time-point method, which is an invasive method that involves drawing a blood sample and sending it off for laboratory analysis. This is a time-consuming method that is often incapable of producing needed results in a timely manner. Other minimally invasive methods such as subcutaneous methods involve the use of a lancet or pin to pierce the skin to obtain a small sample of blood, which is then smeared on a test strip and analyzed by a glucose meter. While these minimally invasive methods may be effective in determining trends in blood glucose concentration, they generally do not track glucose accurately enough to be used for intensive insulin therapy, for example, where inaccuracy at conditions of hypoglycemia could pose a very high risk to the patient.

Electro-chemical biosensors have been developed for measuring various analytes in a substance, such as glucose. An analyte is a substance or chemical constituent that is determined in an analytical procedure, such as a titration. For instance, in an immunoassay, the analyte may be the ligand or the binder, where in blood glucose testing, the analyte is glucose. Electro-chemical biosensors comprise eletrolytic cells including electrodes used to measure an analyte. Two types of electro-chemical biosensors are potentiometric and amperometric biosensors.

Amperometric biosensors, for example, are known in the medical industry for analyzing blood chemistry. These types of sensors contain enzyme electrodes, which typically include an oxidase enzyme, such as glucose oxidase, that is immobilized behind a membrane on the surface of an electrode. In the presence of blood, the membrane selectively passes an analyte of interest, e.g. glucose, to the oxidase enzyme where it undergoes oxidation or reduction, e.g. the reduction of oxygen to hydrogen peroxide. Amperometric biosensors function by producing an electric current when a potential sufficient to sustain the reaction is applied between two electrodes in the presence of the reactants. For example, in the reaction of glucose and glucose oxidase, the hydrogen peroxide reaction product may be subsequently oxidized by electron transfer to an electrode. The resulting flow of electrical current in the electrode is indicative of the concentration of the analyte of interest.

FIG. 1b is a schematic diagram of an exemplary electro-chemical biosensor, and specifically a basic amperometric biosensor. The biosensor comprises two working electrodes: a first working electrode 12 and a second working electrode 14 (the second working electrode is sometimes referred to as the blank electrode). The first working electrode 12 is typically an enzyme electrode either containing or immobilizing an enzyme layer. The second working electrode 14 is typically identical in all respects to the first working electrode 12, except that it may not contain an enzyme layer. The biosensor also includes a reference electrode 16 and a counter electrode 18. The reference electrode 16 establishes a fixed potential from which the potential of the counter electrode 18 and the working electrodes 12 and 14 are established. In order for the reference electrode 16 to function properly, no current must flow through it. The counter electrode 18 is used to conduct current in or out of the biosensor so as to balance the current generated by the working electrodes. The four electrodes together are typically referred to as a cell. During operation, outputs from the working electrodes are monitored to determine the amount of an analyte of interest that is in the blood. Potentiometric biosensors operate in a similar manner to detect the amount of an analyte in a substance.

Currently, significant inaccuracies are present in measuring analytes. Indeed, the electrodes typically have some offset from an accurate measurement of the glucose. For example, when no glucose is present, the current of the working electrode should ideally be equal to the current of the blank electrode, both indicating that no glucose is present. However, oftentimes, this is not the case and thus, presenting a glucose reading having inaccuracies. This may be a particular problem where the patient is, for example, entering surgery, where blood content monitoring is critical.

In light of the above, systems, methods and computer program products are needed to accurately measure levels of analytes in the blood.

### BRIEF SUMMARY OF THE INVENTION

According to one embodiment of the present invention, an analyte monitoring method is disclosed for measuring an analyte concentration utilizing at least two analyte detecting electrodes (working and blank electrode) and calculating the amount of an analyte in a fluid. The measurement of current from the working electrode and current from the blank electrode are used to determine the ratio of current corresponding to the ratio of surface areas of the working electrode and the blank electrode. This ratio is then used to correct any current output from the two electrodes termed “offset” during the analyte measurement process.

In accordance with another embodiment of the present invention, the ratio of the working electrode and blank electrode currents are measured at an applied potential where the electrode current is independent of the analyte to be measured.

In accordance with another embodiment of the present invention, the ratio of the working and blank electrodes is calculated in an electrochemically active region of the analyte to be measured but at a time where the current at the initially applied voltage is significantly greater than the equilibrium current, i.e., in the first few seconds that desired potential is applied to the electrode system.

In accordance with another embodiment of the present invention, a system is disclosed for calculating the amount of an analyte in a fluid using a biosensor. The system includes a biosensor, a controller and a processing unit. The biosensor is capable of sensing an analyte in the fluid and outputting a signal corresponding to an analyte concentration in the fluid, the biosensor comprising a working electrode and a blank electrode. The controller is configured to measure current from each of the electrodes. The processing unit is in communication with the biosensor and is configured to respond to computer instructions to: i) determine a ratio of the working electrode and the blank electrode; and ii) correct an offset using the ratio.

In accordance with yet another embodiment of the present invention, disclosed is a computer program product for calculating the amount of an analyte in a fluid using a biosensor. The computer program product includes a computer usable medium having computer usable program code embodied therein. The computer usable medium includes computer usable program code configured for receiving measurements of current from a working electrode and current from a blank electrode and computer usable program code configured for determining a ratio of the working electrode and the blank electrode. The computer usable medium further includes computer usable program code configured for correcting an offset using this ratio.

In accordance with another embodiment of the present invention, the normalization technique can be extended to a three working electrode system with different surface areas by applying the same principles where a normalization ratio of a first working electrode current over a blank electrode current corrects for first working electrode output with respect to the blank electrode current and a second normalization ration corrects for the second working electrode output with respect to the blank electrode output such that the blank electrode surface area is normalized for each individual working electrode used for an analyte determination.

In accordance with another embodiment of the present invention, using a normalization ratio corresponding to a ratio of electrode surface area are applied to a plurality of working electrodes and a plurality of blank electrodes.

### BRIEF DESCRIPTION OF THE DRAWINGS

Henceforth reference is made to the accompanied drawings and associated text, whereby the present invention is described through given examples and provided embodiments for a better understanding of the invention, wherein:

FIG. 1a is an illustrative block diagram of an analyte monitoring system according to one embodiment of the present invention;

FIG. 1b is a schematic diagram of a four-electrode biosensor of an analyte system in accordance with one embodiment of the present invention;

FIG. 1c is a schematic diagram of a four-electrode biosensor of the analyte system of FIG. 1a;

FIG. 2 is an illustrative block diagram of the analyte monitoring system of FIGS. 1a and 1b;

FIG. 3 is a flow chart of disclosed process according to one embodiment of the present invention;

FIG. 4 illustrates experimental plots of measured current from two sets of working and blank electrodes in accordance with one embodiment of the present invention;

FIG. 5 is a experimental plot of a ratio of the current of a working electrode to the current of the blank electrode of FIG. 4;

FIG. 6 illustrates experimental plots of the normalization current ratio correcting two working electrodes in accordance with one embodiment of the present invention;

FIG. 7 illustrates experimental plots of the current of the working electrode minus the blank electrode;

FIG. 8 illustrates the experimental plots of FIG. 7 corrected by the normalization ratio in accordance with another embodiment of the present invention; and

FIG. 9 illustrates the experimental plots of FIGS. 7 and 8.

### DETAILED DESCRIPTION OF THE INVENTION

The present invention now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Embodiments of the present invention provide systems and methods that allow physicians or other health care workers to monitor a patient using a biosensor, such as an electro-chemical biosensor comprising an electrolytic cell. The electro-chemical biosensor may contain an enzyme capable of reacting with an analyte in a fluid, such as glucose, to generate electrical signals. These signals are sent to a processor, which calculates the amount of substance in the fluid, for example, the blood glucose concentration in blood. The results can then be conveniently displayed for the attending physician. In some embodiments, the biosensor can operate continually when it is installed in the blood vessel, and the results may be seen in real time whenever they are needed. This has the advantage of eliminating costly delays that occur using the conventional method of extracting blood samples and sending them off for laboratory analysis. In some instance, the biosensor is fitted to a catheter, such that it may be placed into the patient's blood stream. In this instance, use of the intravenous biosensor means that the patient does not suffer any discomfort from periodic blood drawing, or experience any blood loss whenever a measurement needs to be taken.

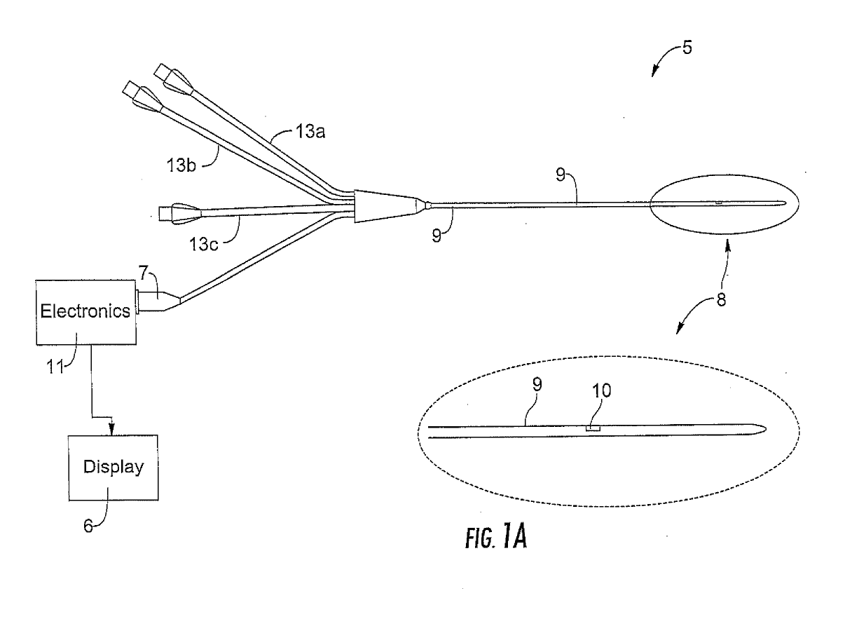

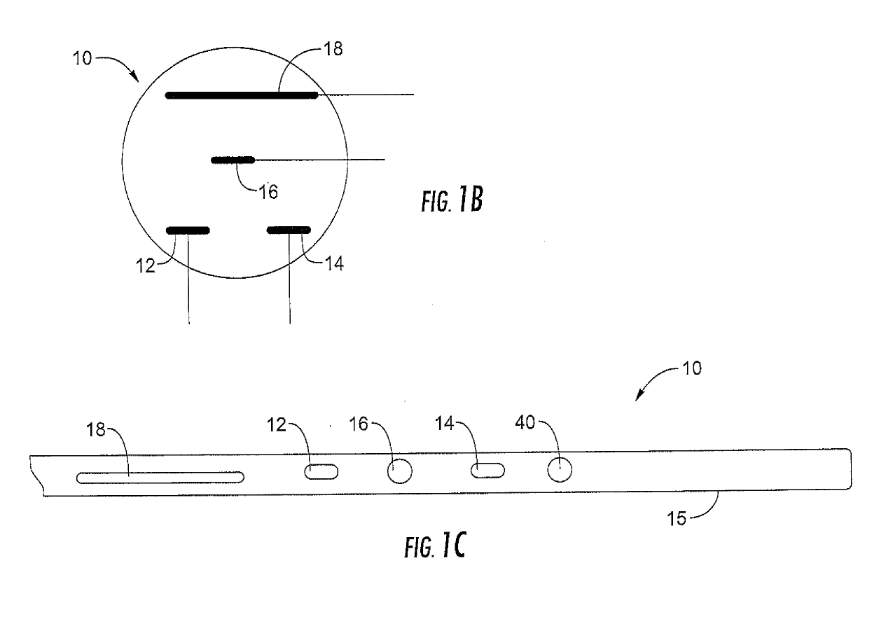

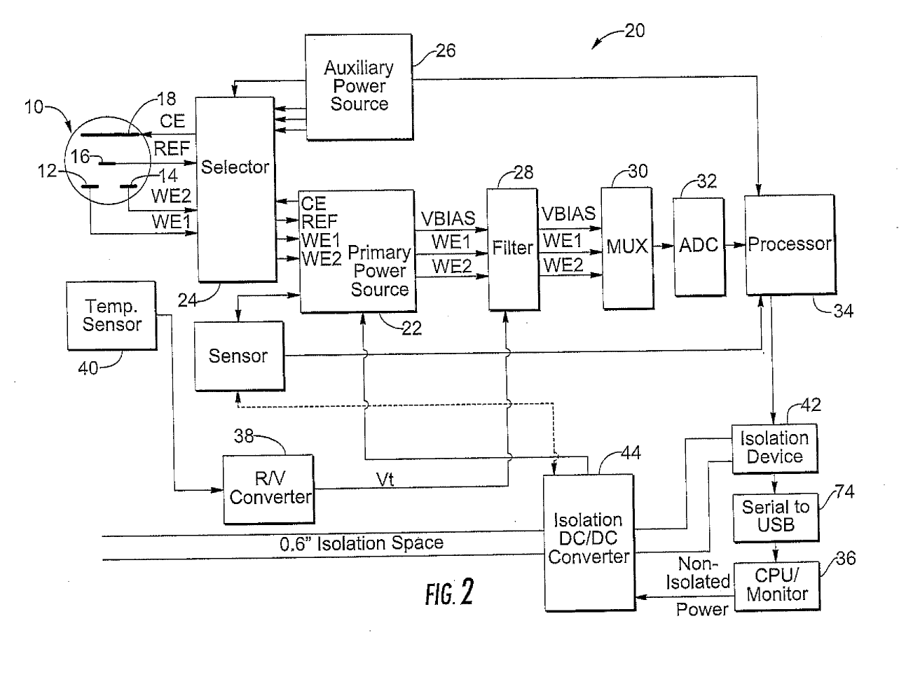

FIG. 1a illustrates an exemplary analyte system 5 according to one embodiment of the present invention. The analyte system 5 includes at least: a biosensor 10, control electronics 11, an electrical connection 7, and a catheter 9. As illustrated in the elliptical area 8 of FIG. 1a, the biosensor 10 is fitted into the catheter 9. A window or opening may be formed in the catheter adjacent to the location of the biosensor. In this configuration, when the catheter is inserted intravenously, the sensor is exposed to the blood stream of the patient for performing analyte concentration measurements, such as glucose concentration measurements.

The control electronics 11 are configured to interact with the biosensor 10 so as to perform analyte concentration monitoring. For example, the control electronics 11 may include components to provide power to the biosensor 10, as well as electronics to receive signals output from the biosensor 10. Additionally, other electronics may be provided to perform signal processing and analog to digital signal conversion. Further, yet other electronics are provided to process the signals output by the biosensor 10 to determine analyte concentration measurements and possibly present such measurements on a display 6 and/or store such measurements via a storage medium (not shown). In the illustrated embodiment, the control electronics 11 are shown as remote from the biosensor 10 and in communication with the biosensor 10 by either wiring or in some embodiments, wireless communication. In some embodiments, some or all of the control electronics 10 can be located more proximate to the biosensor 10, such as at a connector 7 of the catheter 9 (as illustrated in FIG. 1a) or possibly resident with the biosensor 10.

The biosensor 10 is capable of measuring one or more liquid or chemical compositions. In one embodiment, the biosensor 10 measures glucose level in blood by contacting the biosensor 10 with the blood. It should be understood that various other systems (not shown) may be attached to this system, including computer systems, output devices (including a display 6), input devices, and other appropriate devices. For example, although the particular embodiment in FIG. 1a illustrates a catheter 9 having a single electrical connection 7, other embodiments having one or more lumens 13a-c and multiple electrical connections are possible.

It must be understood that the systems and methods of the present invention may be used with any biosensor using a dual electrode measurement system. For example, the systems and methods may be used with electro-chemical biosensors having eletrolytic cells, such as amperometric and potentiometric biosensors containing two or more electrodes used to measure an analyte in a substance, such as glucose in blood, where the analyte measurement is based on a comparison of two or more electrodes of the electrolytic cell.

For example, FIG. 1b is a schematic diagram of an amperometric, four-electrode biosensor 10 which can be used in conjunction with the present invention. In the illustrated embodiment, the biosensor 10 includes two working electrodes: a first working electrode 12 and a second working or blank electrode 14. The first working electrode 12 may be a platinum based enzyme electrode, i.e. an electrode containing or immobilizing an enzyme layer. In one embodiment, the first working electrode 12 may immobilize an oxidase enzyme, such as in the sensor disclosed in U.S. Pat. No. 5,352,348, the contents of which are hereby incorporated by reference. In some embodiments, the biosensor is a glucose sensor, in which case the first working electrode 12 may immobilize a glucose oxidase enzyme. The first working electrode 12 may be formed using platinum, or a combination of platinum and carbon materials. The second working electrode 14 may be substantially identical in all respects to the first working electrode 12, except that it may not contain an enzyme layer. The biosensor 10 further includes a reference electrode 16 and a counter electrode 18. The reference electrode 16 establishes a fixed potential from which the potential of the counter electrode 18 and the working electrodes 12 and 14 may be established. The counter electrode 18 provides a working area for conducting the majority of electrons produced from the oxidation.

The amperometric biosensor 10 operates according to an amperometric measurement principle, where the first working electrode 12 is held at a positive potential relative to the reference electrode 16. In one embodiment of a glucose monitoring system, the positive potential is sufficient to sustain an oxidation reaction of hydrogen peroxide, which is the result of glucose reaction with glucose oxidase. Thus, the first working electrode 12 may function as an anode, collecting electrons produced at its surface that result from the oxidation reaction. The collected electrons flow into the first working electrode 12 as an electrical current. In one embodiment with the first working electrode 12 coated with glucose oxidase, the oxidation of glucose produces a hydrogen peroxide molecule for every molecule of glucose when the working electrode 12 is held at a potential between about +450 mV and about +650 mV. The hydrogen peroxide produced oxidizes at the surface of the first working electrode 12 according to the equation:

H₂O₂→2H⁺+O₂+2e⁻

The equation indicates that two electrons are produced for every hydrogen peroxide molecule oxidized. Thus, under certain conditions, the amount of electrical current may be proportional to the hydrogen peroxide concentration. Since one hydrogen peroxide molecule is produced for every glucose molecule oxidized at the first working electrode 12, a linear relationship exists between the blood glucose concentration and the resulting electrical current. The embodiment described above demonstrates how the first working electrode 12 may operate by promoting anodic oxidation of hydrogen peroxide at its surface. Other embodiments are possible, however, wherein the first working electrode 12 may be held at a negative potential. In this case, the electrical current produced at the first working electrode 12 may result from the reduction of oxygen. The following article provides additional information on electronic sensing theory for amperometric glucose biosensors: J. Wang, “Glucose Biosensors: 40 Years of Advances and Challenges,” Electroanaylsis, Vol. 13, No. 12, pp. 983-988 (2001).

The voltage potential provided by the reference electrode 16 is also provided to the second working or blank electrode 14. As the second working electrode 14 is substantially similar to the first working electrode 12, but for the absence of an enzyme layer, the second working electrode 14 provides and indication of conductivity of both the first and second electrodes structures. As such, by comparing the current output between the first and second working electrodes in response to the potential from the reference electrode, the affects of the enzyme layer on the first working electrode 12 output can be isolated. For example, the current output by the second working or blank electrode 14 may be subtracted from the current output from the first working electrode 12 to there by determine the affects of the enzyme layer's interaction with an analyte. This difference provides an approximation of the amount of the tested analyte in the fluid being monitored.

FIG. 1c illustrates a schematic diagram of an amperometric, four-electrode biosensor 10 according to another embodiment of the present invention. The biosensor 10 is configured to work with the system 5 of FIG. 1a. The biosensor 10 of FIG. 1c includes a first working electrode 14, a second working electrode 12, a blank electrode 16 and a counter electrode 18 and functions similar to the biosensor of FIG. 1b, as previously described. The biosensor 10 of this embodiment further includes a temperature sensor 40, such as a thermo couple; the purpose of which is described later below. The temperature sensor may be located on either the same side or an opposite side of the substrate from the electrodes. The biosensor 10 of FIG. 1c is fabricated on a flexible substrate 15 and is capable of being exposed from the catheter 9 (FIG. 1a) to an in vivo biofluid. Note that traces connecting electronics to each electrode and the temperature sensor are not illustrated in FIG. 1c.

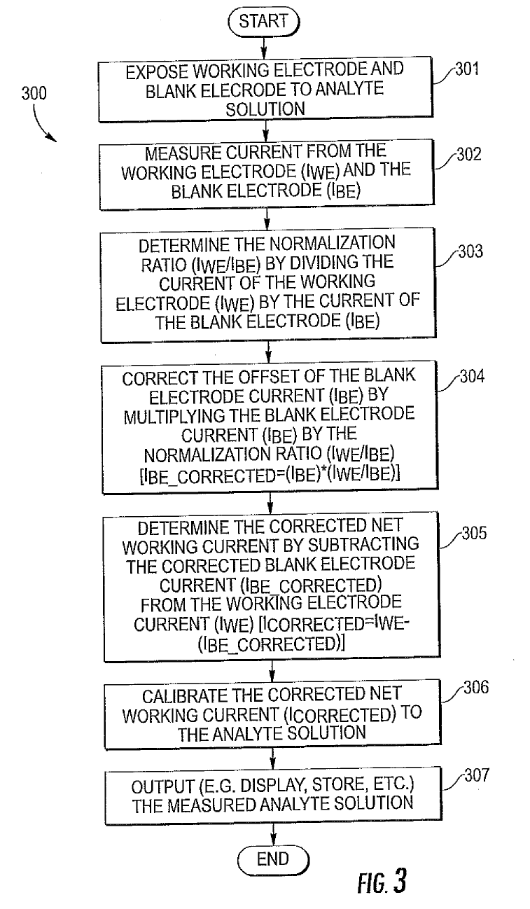

FIG. 2 illustrates a schematic block diagram of a system 20 for operating an electro-chemical biosensor such as an amperometric or potentiometric sensor, such as a glucose sensor. In particular, FIG. 2 discloses a system comprising an amperometric biosensor, such as the one described in FIG. 1b. As more fully disclosed in U.S. patent application Ser. No. 11/696,675, filed Apr. 4, 2007, and titled Isolated Intravenous Analyte Monitoring System, a typical system for operating an amperometric sensor includes a potentiostat 22 in communication with the sensor 10. In normal operation, the potentiostat biases both the electrodes of the sensor and provides outputs regarding operation of the sensor. As illustrated in FIG. 2, the potentiostat 22 receives signals WE1, WE2, and REF respectively from the first working electrode 12, second working or blank electrode 14, and the reference electrode 16. The potentiostat further provides a bias voltage CE input to the counter electrode 18. The potentiostat 22, in turn, outputs the signals WE1, WE2 from the working electrodes 12 and 14 and a signal representing the voltage potential VBIAS between the counter electrode 18 and the reference electrode 16.

A potentiostat is a controller and measuring device that, in an electrolytic cell, keeps the potential of the working electrode 12 at a constant level with respect to the reference electrode 16. It consists of an electric circuit which controls the potential across the cell by sensing changes in its electrical resistance and varying accordingly the electric current supplied to the system: a higher resistance will result in a decreased current, while a lower resistance will result in an increased current, in order to keep the voltage constant.

Another function of the potentiostat is receiving electrical current signals from the working electrodes 12 and 14 for output to a controller. As the potentiostat 22 works to maintain a constant voltage for the working electrodes 12 and 14, current flow through the working electrodes 12 and 14 may change. The difference in the current signals between the working electrodes 12 and 14 indicates the presence of an analyte of interest in blood. In addition, the potentiostat 22 holds the counter electrode 18 at a voltage level with respect to the reference electrode 16 to provide a return path for the electrical current to the bloodstream, such that the returning current balances the sum of currents drawn in the working electrodes 12 and 14.

While a potentiostat is disclosed herein as the first or primary power source for the electrolytic cell and data acquisition device, it must be understood that other devices for performing the same functions may be employed in the system and a potentiostat is only one example. For example, an amperostat, sometimes referred to as a galvanostat, could be used.

As illustrated in FIG. 2, the output of the potentiostat 22 is typically provided to a filter 28, which removes at least some of the spurious signal noise caused by either the electronics of the sensor or control circuit and/or external environmental noise. The filter 28 is typically a low pass filter, but can be any type of filter to achieve desired noise reduction.

In addition to electrical signal noise, the system may also correct analyte readings from the sensor based on operating temperature of the sensor. With reference to FIG. 2, a temperature sensor 40 may be collocated with the biosensor 10. Since chemical reaction rates (including the rate of glucose oxidation) are typically affected by temperature, the temperature sensor 40 may be used to monitor the temperature in the same environment where the working electrodes 12 and 14 of the biosensor are located. In the illustrated embodiment, the temperature sensor may be a thermistor, resistance temperature detector (RTD), or similar device that changes resistance based on temperature. An R/V converter 38 may be provided to convert the change in resistance to a voltage signal Vt that can be read by a processor 34. The voltage signal Vt represents the approximate temperature of the biosensor 10. The voltage signal Vt may then be output to the filter 28 and used for temperature compensation.

As illustrated in FIG. 2, a multiplexer may be employed to transfer the signals from the potentiostat 22, namely 1) the signals WE1, WE2 from the working electrodes 12 and 14; 2) the bias signal VBIAS representing the voltage potential between the counter electrode 18 and the reference electrode 16; and 3) the temperature signal Vt from the temperature sensor 40 to the processor 34. The signals are also provided to an analog to digital converter (ADC) 32 to digitize the signals prior to input to the processor.

The processor uses algorithms in the form of either computer program code where the processor is a microprocessor or transistor circuit networks where the processor is an ASIC or other specialized processing device to determine the amount of analyte in a substance, such as the amount of glucose in blood. The results determined by the processor may be provided to a monitor or other display device 36. As illustrated in FIG. 2 and more fully described in U.S. patent application Ser. No. 11/696,675, filed Apr. 4, 2007, and titled Isolated Intravenous Analyte Monitoring System, the system may employ various devices to isolate the biosensor 10 and associated electronics from environmental noise. For example, the system may include an isolation device 42, such as an optical transmitter for transmitting signals from the processor to the monitor to avoid backfeed of electrical noise from the monitor to the biosensor and its associated circuitry. Additionally, an isolated main power supply 44 for supplying power to the circuit, such as an isolation DC/DC converter.

The processor 34 analyzes the difference in output between the first and second working electrodes 12 and 14. This difference indicates the analyte level in the fluid.

A typical biosensor measures current from the working electrode and current from the blank electrode and a net working current is then determined by subtracting the blank electrode current from the working electrode current. This net working current is directly proportional to the analyte concentration values and is thus used to determine analyte concentration. As such, the accuracy of the net working current is important. However, typically the measured net working current is somewhat inaccurate because the working electrode current and the blank electrode current in the absence of an electroactive substance are not equal. This differential current in the absence of electroactive substances is termed “offset”. It has been determined that such offset is caused, in part, by the working electrode having a surface area unequal to the surface area of the blank electrode. In accordance with some embodiments of the present invention, this offset can be corrected by applying a normalization ratio (which will be further described below) to the blank electrode current (or working electrode current) prior to calculating the net working electrode current. Accordingly, the net working electrode current can be adjusted to compensate for surface area differences between the electrodes resulting in a minimization of offset. A more detailed description of such process is provided below.

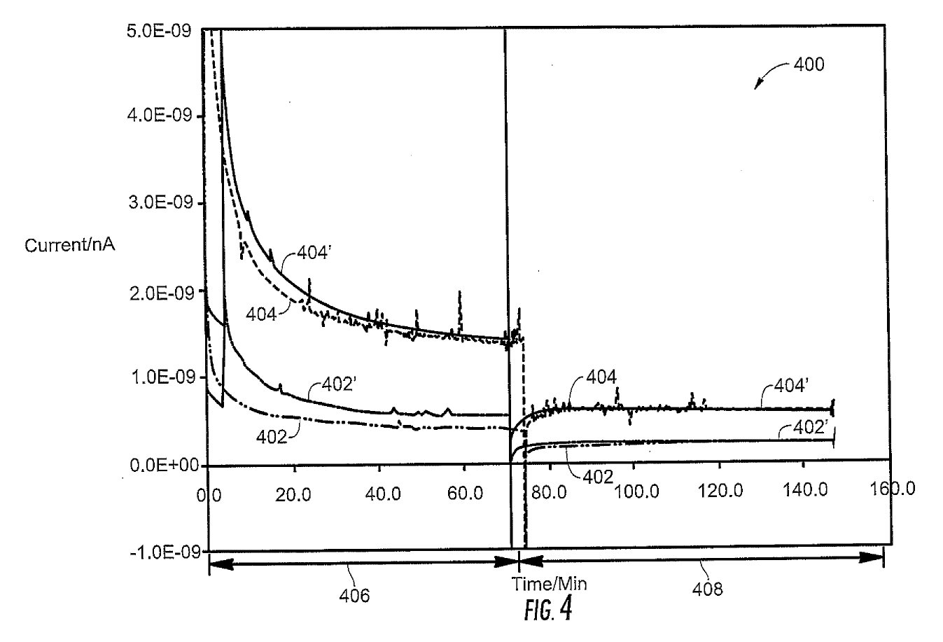

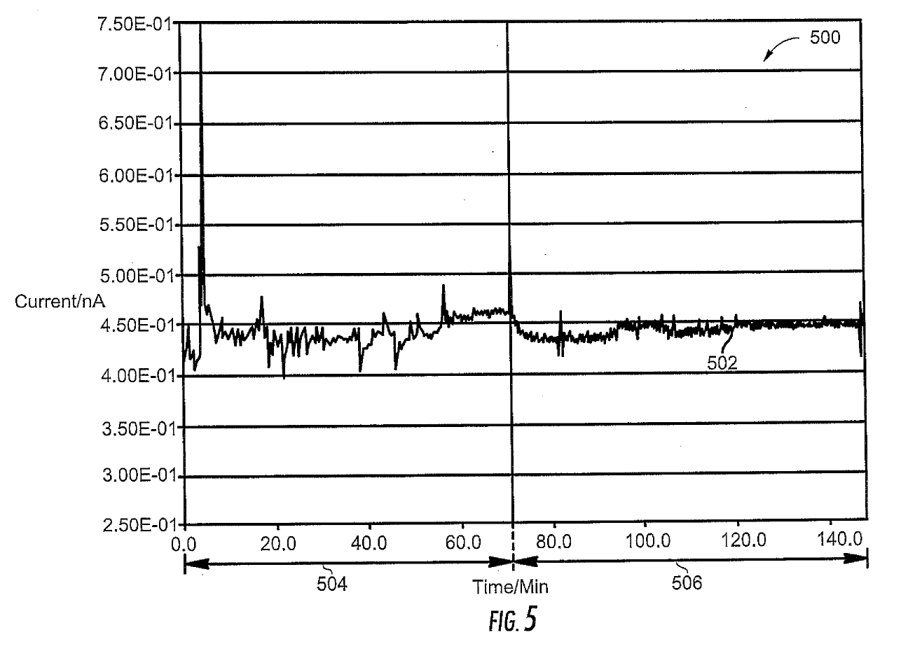

A general description of the method 300 according to one embodiment will now be described with reference to the flow chart of FIG. 3. First, the working electrode and the blank electrode are exposed to a solution having an analyte or no analyte (block 301). The current is measured from the working electrode and the blank electrode (block 302). The normalization ratio (Nₒ=I_{WEo}/I_{BEo}) is determined by dividing the current (I_{WE}) of the working electrode by the current (I_{BE}) of the blank electrode (block 303). Such ratio Nₒ corresponds to the ratio of surface areas of the working electrode and the blank electrode. The blank electrode current for subsequent measurements is corrected by multiplying the blank electrode current by the normalization ratio (I_{BEc}=I_{BE}*Nₒ) (block 304). Then, the corrected net working current (I_{c}) is calculated by subtracting the corrected blank electrode current from the working electrode current [I_{c}=I_{WE}−(I_{BE})(Nₒ)] (block 305). It should be noted that the normalization ratio is calculated using one set of points on the working electrode current plot and blank electrode plot, but is applied to all points on working electrode current plot and blank electrode plot. After determining the corrected net working current (I_{c}), the corrected net working current (I_{c}) is calibrated to reflect analyte solution concentration values (block 306). Then, the analyte solution concentration values are outputted to a device (such as a display monitor, a file on a computer, memory in a computer, a print out, to any other medium) (block 307). This method 300 will be described in more depth with regard to FIGS. 4-9 below.

FIGS. 4-9 illustrate results of various experimental tests confirming that applying a normalization ratio can minimize offset of net working current. A description of how the experiments were set up will now be described. First, each sensor has a blank electrode and working electrode, where the working electrode's surface area was masked to be about 50% of the blank electrode's surface area. The working and blank electrodes of each sensor were exposed to a solution having no analyte solution. Approximately −0.85 V was applied to the working and blank electrodes via the reference electrode for about 70 minutes, which is defined as the “run-in period.” After the run-in period, the voltage (−0.85V) was changed to −0.7 V and thus, applied to the working and blank electrodes as normal operating voltage for the sensor. Although, the experiments below have been performed using the above parameters, it should be understood that any of these parameters may be varied or changed and still be consistent with embodiments of the present invention.

FIG. 4 illustrates two experimental plots of measured working electrode current (I_{WE}) 402, 402′ and measured blank electrode current (I_{BE}) 404, 404′ for two exemplary sensors. As mentioned above, each sensor has a working electrode with a surface area that is approximately 50% smaller than the surface area of the blank electrode. As illustrated in both experimental tests, the working electrode current (I_{WE}) 402, 402′ was reduced (lower in value) from the blank electrode current (I_{BE}) 404, 404′ because of the reduced surface area of the working electrodes. Ideally, for electrodes in which both working and blank electrode surface areas are equal in a solution where no analyte is present, the working electrode current 402, 402′ should be equal to the blank electrode current 404. It should be noted that the run-in period 406 is only used to condition the sensor and the period 408 after the nm-in period 406 is used in measuring analyte concentration.

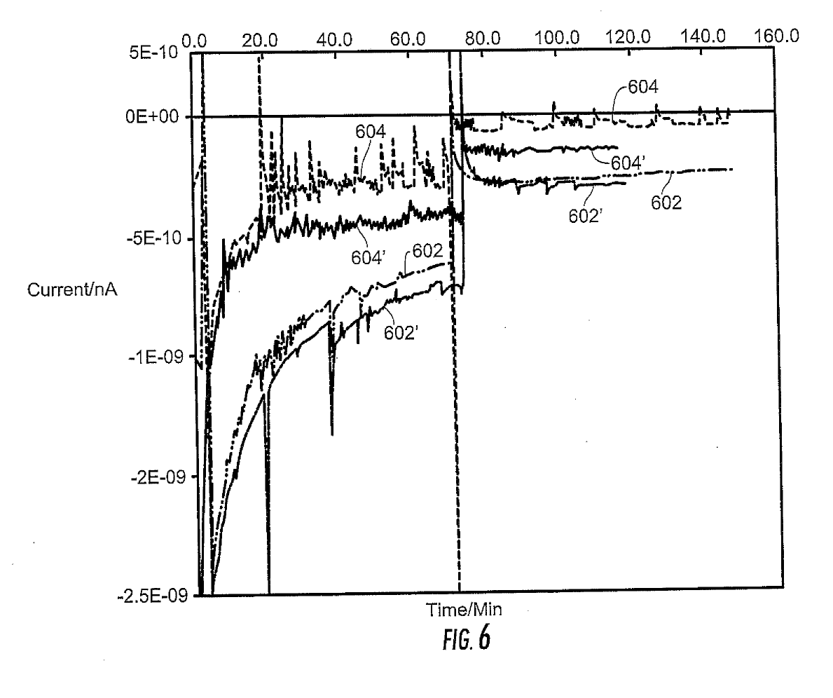

After measuring the working electrode current (I_{WE}) 402, 402′ and the blank electrode current (I_{BE}) 404, 404′, a normalization ratio (Nₒ) 502 was calculated. FIG. 5 illustrates the normalization ratio 502 plotted versus time (min.). During the run-in period 504, the normalization ratio 502 was somewhat variable, but on average was approximately the same as after the run-in period 506. After the run in period 506, the normalization ratio 502 leveled off to a relatively constant value. As illustrated, the normalization ratio 502 is about 0.45 or 45%, which is approximately equal to the ratio of the working electrode surface area to the blank electrode surface area. Thus, the normalization ratio 502 allows one to compensate for differences in surface area of the electrodes.

Referring briefly back to FIG. 3, FIG. 3 describes that the net working current is corrected by multiplying the normalization ratio times the blank electrode current and then using this value to calculate the net working current. However, an alternate embodiment includes correcting the net working current by diving the working electrode current by the normalization ratio and determining the net working current by subtracting the blank electrode current from the corrected working electrode current [I_{Corrected}=I_{WE}/(I_{WE}/I_{BE})−(I_{BE})].

The concepts described above can be implemented utilizing similar algebraic manipulations to either normalize the working electrode output or the blank electrode output, and can be envisioned by utilizing the normalization ratio Nₒ and then normalizing the blank electrode output, or similarly as using an inverse of the normalization ratio (1/Nₒ) and normalizing the working electrode output.

FIGS. 6-8 illustrate the method 300 presented in FIG. 3, where the corrected net working current is calculated by multiplying the normalization ratio times the blank electrode current and subtracting this from the working electrode current. Each of FIGS. 6-8 illustrates a plot of the net working current (I_{Net}) of two or more sensors. The net working current (I_{Net}), as previously mentioned, means the working electrode current minus the blank electrode current (I_{Net}=I_{WE}−I_{BE}). FIGS. 6-8 also illustrate a control net working current (I_{NET}_{_{—}}_{control}) and a reduced-area net working current (I_{Net}_{_{—}}_{reduced}_{_{—}}_{area). The control net working current (I}_{NET}_{_{—}}_{control}) refers to a net working current where the working electrode of the sensor being measured has a surface area approximately equal to the surface area of the blank electrode. The reduced-area net working current (I_{Net}_{_{—}}_{reduced}_{_{—}}ₐᵣₑₐ) refers to a net working current where the working electrode of the sensor being measured has a surface area reduced relative to the surface area of the blank electrode.

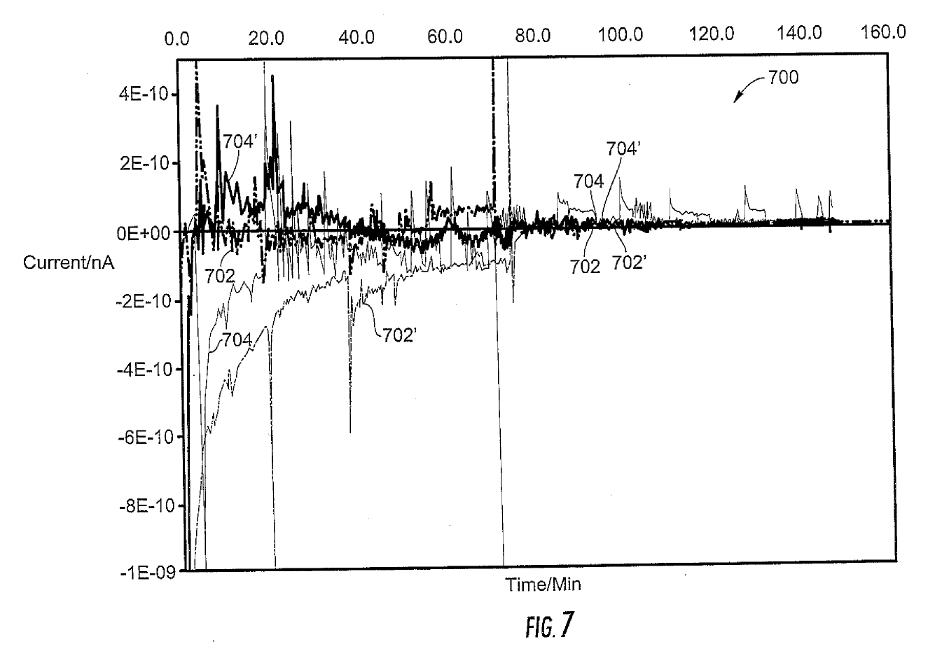

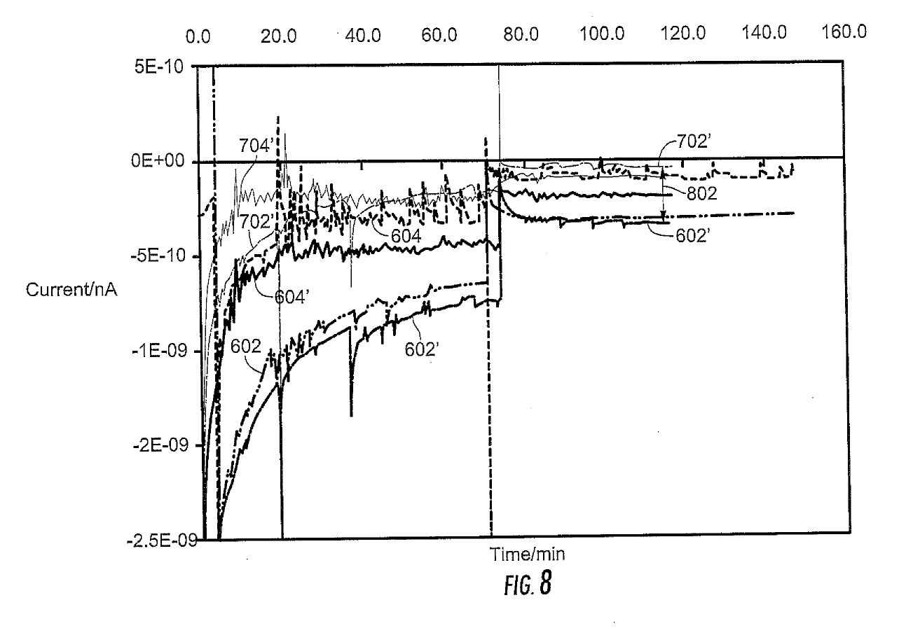

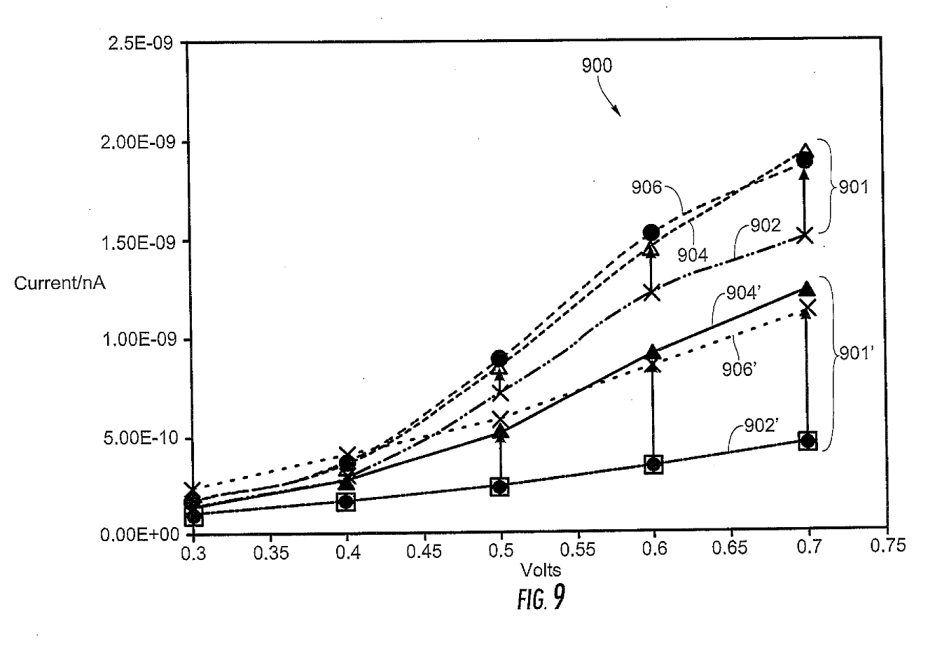

Generally, FIG. 6 illustrates plots 600 of a reduced-area net working current (I_{Net}_{_{—}}_{reduced}_{_{—}}ₐᵣₑₐ) as well as a plot of a control net working current (I_{NET}_{_{—}}_{control}); FIG. 7 illustrates plots 700 of the corrected net working current (I_{NET}_{_{—}}_{control}); and FIG. 8 illustrates the plots of FIGS. 6 and 7 on a single graph.

FIG. 6 illustrates plots 600 of net working current versus time (min.) for four sensors: i) a plot of reduced-area net working current (I_{Net}_{_{—}}_{reduced}_{_{—}}ₐᵣₑₐ) 602, 602′ for two of the sensors, and ii) a plot of a control net working current (I_{NET}_{_{—}}_{control}) 604, 604′ for the two other sensors. The I_{Net}_{_{—}}_{reduced}_{_{—}}ₐᵣₑₐ 602, 602′ has not been corrected in FIG. 6. As illustrated, the (I_{Net}_{_{—}}_{reduced}_{_{—}}ₐᵣₑₐ) 602, 602′ is substantially lower (or offset) than the I_{NET}_{_{—}}_{control} 604, 604′ due to the difference in surface areas between the working and blank electrodes. The greater the difference in surface areas between the electrodes of the sensor, the greater the offset in the reduced-area net working current I_{Net}_{_{—}}_{reduced}_{_{—}}ₐᵣₑₐ 602, 602′ relative to the control net working current I_{NET}_{_{—}}_{control} 604, 604′. This proves that the surface area of the working electrode relative to the blank electrode creates an offset in measured net working current. Such offset should be corrected in order to avoid inaccuracies in measuring analyte concentration of a solution. As previously discussed, the normalization ratio Nₒ provides for such correction as will be described below with regard to FIG. 7.

FIG. 7 illustrates the experimental plots of FIG. 6 corrected by the normalization ratio Nₒ calculated for each respective electrode. Specifically, FIG. 7 illustrates an experimental plot for each of the four sensors of FIG. 6: i) a plot of corrected I_{Net}_{_{—}}_{reduced}_{_{—}}ₐᵣₑₐ 702, 702′ for two sensors, and ii) a plot of corrected I_{NET}_{_{—}}_{control} 704, 704′ for the two control sensors. Each of the plots of FIG. 7 has been corrected by using the normalization ratio Nₒ 502 for each respective electrode, as described above via in the embodiment of FIG. 3. In other words, each plot in FIG. 7 is a plot of [I_{WE}−(I_{BE}*Nₒ)] versus time, where Nₒ is unique for each electrode. As illustrated in FIG. 7, after the run-in period, the corrected I_{Net}_{_{—}}_{reduced}_{_{—}}ₐᵣₑₐ 702, 702′ has been normalized and measures close to a zero reading. This is an accurate reading since no analyte solution was introduced into the measured solution. Also, the error in the corrected I_{Net}_{_{—}}_{reduced}_{_{—}}ₐᵣₑₐ 702 is in the picoamp range (shown in FIG. 7) as opposed to the I_{NET}_{_{—}}_{control} error being in the nanoamp range (shown in FIG. 6). It should be noted that the I_{NET}_{_{—}}_{control} corrected using the normalization ratio also shows improvement over the plots 604 and 604′ even though the surface areas of the working electrode and blank electrodes are equal by design yet still have a small offset that can be corrected for.

FIG. 8 illustrates the experimental plots 600, 700 of FIGS. 6 and 7 plotted on a single graph 800. As illustrated, the offset 802 of the reduced-area net working current I_{Net}_{_{—}}_{reduced}_{_{—}}ₐᵣₑₐ 602′ is adjusted using the normalization ratio Nₒ 502, resulting in a corrected I_{Net}_{_{—}}_{reduced}_{_{—}}ₐᵣₑₐ 702′ with a nearly zero offset. This corrected I_{Net}_{_{—}}_{reduced}_{_{—}}ₐᵣₑₐ 702′ is then translated via the control electronics 11 to determine an analyte concentration value. The analyte concentration value is then outputted to an output device of the biosensor, such as via a display device.

The method of normalizing the blank current using a normalization ratio Nₒ′ can be applied to electrodes when using different applied voltages as illustrated in FIG. 9. Specifically, FIG. 9 shows that the net working current is adjusted by first correcting the working electrode current, and this corrected working electrode current is used in calculating the net working current. FIG. 9 illustrates results of two experimental tests 901 and 901′, each experimental test measuring three sets of currents: i) working electrode current 902 or 902′, respectively, ii) blank electrode current 904 or 904′, respectively, and iii) corrected working electrode current 906 or 906′, respectively. Each electrode was exposed to a solution having no analyte solution. As previously described, the normalization ratio is first calculated at one voltage by dividing the working electrode current 902 or 902′ by the blank electrode current 904 or 904′, respectively. As illustrated, this normalization ratio (calculated at one voltage) is then used to shift the data point at that particular voltage of the measured working electrode current 902 or 902′, resulting in a plot of a corrected working electrode current 906 or 906′. This is repeated for each applied voltage. Specifically, the entire plot of the measured working electrode current 902, 902′ is corrected by dividing the measured working electrode current 902, 902′ by the normalization ratio for each voltage, as the normalization ratio is dependent to some degree on the applied voltage, especially near the region of the open circuit potential, where no electrochemistry takes place, resulting in the corrected working electrode current 906, 906′. As shown, the corrected working electrode current 906, 906′ closely approximates the blank electrode current 904, 904′. Accordingly, the corrected working electrode current 906, 906′ is accurate based on the assumption that the blank electrode current 904, 904′ closely approximates a working electrode measuring a solution absent of analyte. The corrected working electrode current 906, 906′ is then used to determine the corrected net working current by subtracting the blank electrode current from the corrected working electrode current dependent on the voltage used for the analysis desired.

The above embodiments describe determining the normalization ratio Nₒ through calculations involving the currents of the working electrode and blank electrode. Such normalization ratios correspond to or represent the ratio of the surface areas of the individual electrodes in a sensor configuration. However, it should be understood that the normalization ratio could be determined by any other manner. For example, the normalization ratio could be determined by accurately measuring and dividing the electrodes surface areas according to another embodiment of the present invention. Other methods to determine a ratio which corresponds to the ratio of the surface areas of the working and blank electrodes may also be employed.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other changes, combinations, omissions, modifications and substitutions, in addition to those set forth in the above paragraphs, are possible. Those skilled in the art will appreciate that various adaptations and modifications of the just described embodiments can be configured without departing from the scope and spirit of the invention. Therefore, it is to be understood that, within the scope of the appended claims, the invention may be practiced other than as specifically described herein.

That which is claimed:

1. A method of calculating the amount of an analyte in a fluid using a biosensor comprising:
providing said biosensor comprising a blank electrode and a working electrode;
receiving measurements of current from said working electrode and current from said blank electrode;
determining a normalization ratio corresponding to a ratio of the surface areas of said working electrode and said blank electrode; and
correcting an offset between the two electrodes using said normalization ratio.

2. The method of claim 1, wherein said determining a normalization ratio comprises dividing said working electrode current by said blank electrode current at a first time, and wherein said correcting an offset using said normalization ratio comprises determining a corrected blank electrode current by multiplying said blank electrode current at time subsequent to said first time by said normalization ratio.

3. The method of claim 2, further comprising calculating a net working current by subtracting said corrected blank electrode current from said working electrode current.

4. The method of claim 3, further comprising calibrating said net working current to an analyte concentration value.

5. The method of claim 4, further comprising outputting said calibrated net working current.

6. The method of claim 5, wherein said outputting occurs at a display for a sensor.

7. The method of claim 1, further comprising:
calculating a net working current by subtracting said blank electrode current from a corrected working electrode current,
wherein said determining a normalization ratio comprises dividing said working electrode current by said blank electrode current, and
wherein said correcting said offset using said normalization ratio comprises determining said correcting working electrode current by dividing said working electrode current by said normalization ratio.

8. The method of claim 1, wherein said determining a normalization ratio comprises calculating said normalization ratio by dividing said working electrode current by said blank electrode current.

9. The method of claim 1, wherein said determining a normalization ratio comprises:
measuring said working electrode surface area and said blank electrode surface area; and
calculating said normalization ratio by dividing said working electrode surface area by said blank electrode surface area.

10. The method of claim 1, wherein said receiving measurements of current from a working electrode and current from a blank electrode comprises:
exposing said working electrode and said blank electrode to a solution; and
measuring said working electrode current and said blank electrode current in response to exposing said working electrode and said blank electrode to said solution.

11. The method of claim 10, wherein said solution contains substantially no analyte solution.

12. The method of claim 10, wherein said solution comprises glucose.

13. The method of claim 1, wherein the normalization ratio is determined prior to completing a run-in of the electrode.

14. The method of claim 1, further comprising:
determining a second normalization ratio for a second working electrode current, the second normalization ratio corresponds to a ratio of the surface areas of said second working electrode and said blank electrode; and
correcting an offset between said second working electrode current and said blank electrode current using said second normalization ratio.

15. A method of calculating the amount of an analyte in a fluid using a biosensor, the method comprising:
providing said biosensor comprising a working electrode and a blank electrode;
determining a ratio of measured current from said working electrode and current measured from said blank electrode; and
calculating in said biosensor a net working current by subtracting said blank electrode current from a corrected working electrode current, said corrected working electrode current is determined by multiplying said measured working electrode current by said ratio.

16. The method of claim 15, wherein said ratio is determined by dividing said working electrode current by said blank electrode current.

17. The method of claim 15, wherein said corrected net working current is used to determine an analyte concentration.

18. A system for calculating the amount of an analyte in a fluid using a biosensor comprising:
a biosensor capable of sensing an analyte in the fluid and outputting a signal corresponding to an analyte concentration in the fluid, said biosensor comprising a working electrode and a blank electrode;
a controller configured to measure current from each of said electrodes; and
a processing unit in communication with said biosensor, wherein said processing unit is configured to respond to computer instructions to:
determine a normalization ratio corresponding to a ratio of the surface areas of said working electrode and said blank electrode; and
correct a differential current offset using said normalization ratio.

19. The system of claim 18, wherein said processing unit is further configured to calculate a net working current by subtracting a normalized blank electrode current from said working electrode current, said normalized blank electrode current calculated by multiplying said measured blank electrode current by said normalization ratio.

20. The system of claim 18, further comprising an output device connected to said processing unit in order to display values corresponding to an analyte concentration value.

21. The system of claim 18, wherein said processing unit is configured to determine said ratio by dividing said working electrode current by said blank electrode current.

22. The system of claim 18, wherein said processing unit is further configured to calibrate the measured output signal to an analyte concentration value.

23. A computer program product for calculating the amount of an analyte in a fluid using a biosensor, said computer program product comprising:
a computer usable medium having computer usable program code embodied therein, the computer usable medium comprising:
computer usable program code configured for receiving measurements of current from a working electrode and current from a blank electrode;
computer usable program code configured for determining a ratio of said working electrode and said blank electrode; and
computer usable program code configured for correcting an offset of said net working current by multiplying said ratio times said blank electrode current resulting in a corrected blank electrode current.

24. The computer program product of claim 23, said computer usable medium further comprising computer usable program code configured for calculating a net working current by subtracting said corrected blank electrode current from said working electrode current.
